SEQUENCE LISTING (4 sequences)

SEQ ID NO: 1 - DNA, 23 bp, Artificial Sequence
  Synthetic upstream primer for AT1R
gcttcaacctctacgccagtgtg

SEQ ID NO: 2 - DNA, 24 bp, Artificial Sequence
  Synthetic downstream primer for AT1R
cagccagatgatgatgcaggtgac

SEQ ID NO: 3 - DNA, 23 bp, Artificial Sequence
  Synthetic upstream primer for Beta-actin
ggagattactgccctggctccta

SEQ ID NO: 4 - DNA, 24 bp, Artificial Sequence
  Synthetic downstream primer for Beta-actin
gactcatcgtactcctgcctgctg

(19) United States
(12) Patent Application Publication
(10) Pub. No.: US 2024/0252576 A1
(43) Pub. Date: Aug. 1, 2024

(54) Application Method of Radix Paeoniae Alba and Dendrobium Officinale for Preparing Drug for Treatment of Hypertension

(71) Applicant: Hangzhou Danhe Pharmaceutical Co., Ltd., Hangzhou, Zhejiang Province (CN)

(72) Inventors: Renzhao WU, Hangzhou (CN); Yue WU, Hangzhou (CN); Yu CHEN, Hangzhou (CN)

(21) Appl. No.: 17/255,555

(22) PCT Filed: May 29, 2019
(86) PCT No.: PCT/CN2019/088938

§ 371 Date: Dec. 23, 2020

(30) Foreign Application Priority Data
Jul. 11, 2018 (CN) 201810756180.4

(51) Int. Cl. A61K 36/65; A61K 9/16; A61K 36/8984; A61P 9/12

(52) U.S. Cl. CPC A61K 36/65; A61K 9/1688; A61K 36/8984; A61P 9/12; A61K 2236/15; A61K 2236/331; A61K 2236/333

(57) ABSTRACT

Disclosed is an application method of radix paeoniae alba and dendrobium officinale for preparing a drug for treatment of hypertension, comprising: grinding radix paeoniae alba into powder and performing alcohol extraction, carrying out water extraction on the residue after alcohol extraction, and merging the concentrated extraction solutions; and grinding the dendrobium officinale into powder, adding the merged extraction solutions, and adding auxiliary materials to prepare a drug for treatment of hypertension. According to the present invention, the drug prepared by adding dendrobium officinale into extracts of radix paeoniae alba which is subjected to the alcohol-water extraction method has prominent effect in treating hypertension, and the antihypertensive effect of the drug is superior to that of western medicine irbesartan. Compared with the conventional water extraction, the alcohol-water extraction method of radix paeoniae alba reduces component loss, so as to improve the effectiveness, which promotes the complete utilization of the radix paeoniae alba by the human body in a process of oral medication, and the curative effect is improved. The drug has a selective adjustment function on people with high blood pressure and do not influence normal blood pressure.

### BACKGROUND OF THE PRESENT INVENTION

### Field of Invention

The present invention relates to the technical field of medicine preparation, and in particular, to an application method of Radix Paeoniae Alba and Dendrobium Officinale for preparing drug for treatment of hypertension.

### Description of Related Arts

Hypertension is a common high incidence disease, and has serious harm to important organs such as heart, brain, kidney, and etc. Western medicine for treating hypertension has the lack of regulating physique in addition to adjusting blood pressure (hypotensively). The hypotensive treatments of modern medicine are all treatments of the symptoms. Currently commonly used antihypertensive agents include calcium ion channel blockers (CCB), angiotensin converting enzyme inhibitors (ACEI), angiotensin II receptor blockers (ARB), diuretics, and beta blockers, which are not yet capable of completely preventing the progression of hypertension and its secondary lesions. Most hypertension patients require 2 or more hypotensive drugs to achieve the target blood pressure, or the target blood pressure is difficult to reach, and most patients need to take the medicine lifelong. The curative effect of the medicine is gradually reduced due to the long course of treatment, and the toxic and side effects are generated, which problems are more prominent as the treating is prolonged. There remains an urgent need for new, more sophisticated therapeutic drugs for high blood pressure treatment.

At present, the traditional Chinese medicine conditioning method is somehow helpful for high blood pressure, but it is not fast enough in blood pressure reduction after medicine taking, and the reduced extent is not obvious enough, too. The traditional Chinese medicine compound also includes too many medicines, which can render mutual containment or counteracting effects, so that the actual curative effect of the traditional Chinese medicine compound become limited. Besides, the traditional Chinese medicine composition, the extraction, the preparation process, and the like are not optimized through systematic optimization, causing the traditional Chinese medicine compound be difficult to be valued in the practical application in clinic. There has not been traditional Chinese hypotension medicine introduced into clinical practical application. Certain of the traditional Chinese medicinal materials with relatively high hypotensive curative effect are selected and processed with some common extraction processes. The extraction process optimization study is also carried out, so as to further enhance the hypotensive curative effect. This is an important direction for the development of traditional Chinese hypotensive medicine, while the workload is huge.

A China patent “Application of Dendrobium Officinale in Preparation of Drugs for Treating Hypertension” (Application No. 201710855728.6) mentioned that Dendrobium Officinale powder has a hypotensive curative effect when used as a medicament for treating hypertension (having a hypotensive effect after 24 hours). However, most traditional Chinese medicines have to work through compatibility and are difficult to break through in hypotensive efficacy. This is also a major cause of the traditional Chinese medicine to reduce blood pressure into clinical practical applications over the years. Through experiments, it is known that most of the traditional Chinese medicine used in a single-flavor application manner can provide a certain hypotensive effect, but provide a lower hypotensive curative effect when being used in a compatible application unexpectedly. The compatibility of the traditional Chinese medicine and the process optimization study are both important directions for improving the traditional Chinese medicine hypotensive curative effect, but also require huge workload.

Radix Paeoniae Alba is a traditional Chinese medicine commonly used for nourishing blood, and Dendrobium Officinale is a traditional Chinese medicine commonly used for nourishing yin and clearing heat. Traditional, both of them are usually applied through decocting with water. Under normal usage conditions, when there is only limited hypotension effect, both Radix Paeoniae Alba and Dendrobium Officinale have not been made into prepared Chinese medicine for hypertension. Traditional Chinese medicinal professionals have not used them in the drug matching therefor, too.

### SUMMARY OF THE PRESENT INVENTION

Technical issues to be solved by the present invention include to overcome the deficiencies and prejudice in the prior art, and to provide an application method of Radix Paeoniae Alba and Dendrobium Officinale for preparing drug for treatment of hypertension.

In order to solve the technical issues, a solution of the present invention is:
to provide an application of Radix Paeoniae Alba and Dendrobium Officinale in preparation of drugs for treatment of hypertension, including the following steps:
(1) 8 servings of Dendrobium Officinale and 2-32 servings of Radix Paeoniae Alba according to the requirements of Chinese Pharmacopoeia (2015), wherein serving refers to a serving by weight;
(2) grinding the Radix Paeoniae Alba into powder and carrying out alcohol extraction: adding 7 times of the raw medicine amount of 70% ethanol for reflux extraction for 1 hour, or leaching at 60° C. for 24 hours, wherein the Radix Paeoniae Alba is totally extracted twice, and the ethanol is collected;
(3) performing water extraction treatment on the alcohol extracted residual: extracting twice with hot water at 100° C., and combining the two extracting solutions; or extracting by using hot water at a gradient temperature twice and combining the two extracting solutions, wherein the water temperature is 100° C. for the first water extraction, and the water temperature is 121° C. for the second water extraction, wherein the water extraction time of the two times is 1 hour each;
(4) respectively concentrating the alcohol and water extracting solutions and combining the concentrated extracting solutions; and
(5) grinding the Dendrobium Officinale into powder, and adding into the combined extracting solution; and adding auxiliary materials according to a pharmaceutically conventional processing method so as to prepare a traditional Chinese medicine tablet, suspension particles, pill, powder, oral liquid or capsule to provide the medicine for treating hypertension.

The present invention further provides a using method of the above prepared traditional Chinese medicine for treating hypertension, which is taking the prepared traditional Chinese medicine tablet, suspension particles, pill, powder, oral liquid or capsule orally, wherein the daily dosage of an adult is equivalent to 2.5 g to 40 g of the raw medicine material.

The medicine (drug) for treatment of hypertension prepared by the above method can also be used as a supplement and health-care food, which method is taking the prepared traditional Chinese medicine tablet, suspension particles, pill, powder, oral liquid or capsule as a supplement and health-care food orally, wherein the daily dosage of an adult is equivalent to 2.5 g to 40 g of the raw medicine material.

In the medicine (drug) for treatment of hypertension according to the present invention, the weight servings of the Dendrobium Officinale and the Radix Paeoniae Alba are 4:3, wherein it is orally taken when the medicine is used, and the daily dosage of an adult is equivalent to 7 g to 14 g of the raw medicine material (selected for serving as the main common hypotensive drug).

In the medicine for treatment of hypertension according to the present invention, the weight servings of the Dendrobium Officinale and the Radix Paeoniae Alba are 4:1, wherein it is orally taken when the medicine is used, and the daily dosage of an adult is equivalent to 5 g to 15 g of the raw medicine material (selected when the required hypotensive peak value is not too low, the blood pressure fluctuation is low, or the blood pressure is to be maintained).

In the medicine for treatment of hypertension according to the present invention, the weight servings of the Dendrobium Officinale and the Radix Paeoniae Alba are 1:4, wherein it is orally taken when the medicine is used, and the daily dosage of an adult is equivalent to 5 g to 15 g of the raw medicine material (selected when requires fast hypotensive effect and short duration).

Implementation principles of the present invention:

According to the theory and practice of traditional Chinese medicine (Reference: PRC Pharmacopoeia, 2015), the effect of Dendrobium Officinale is benefiting stomach, promoting salivation, nourishing yin and clearing heat. It can be used for hot disease and supporting for injury, dry mouth and restlessness, deficiency of stomach yin, less eating and vomiting, deficiency heat after disease, yin deficiency and rich fire, bone steaming and hot fatigue, weak eyesight, and soft and weak muscles and bones. The Radix Paeoniae Alba has the properties of nourishing blood, regulating menstruation, astringing yin and stopping sweat, softening liver and relieving pain, and calming liver's yang. It is used for blood deficiency withering, irregular menstruation, spontaneous perspiration, sweating, pain, abdominal pain, limb pain, headache and dizziness.

The use of the two traditional Chinese medicines in the treatment of hypertension is that the Radix Paeoniae Alba is not directly related to the modern hypertension disease, and its curative effect is insufficient if used along according to the traditional Chinese medicine. Dendrobium Officinale has a use for hypertension treatment, but has a relatively slow effect and has no scale application. The Dendrobium Officinale only has the effects of nourishing yin and clearing heat, rather than the effects of nourishing blood, softening liver, and calming liver's yang. On the other hand, the Radix Paeoniae Alba only has the effects of nourishing blood and calming the liver, but is weak in yin-nourishing and heat-clearing effects. Either of them is not ideal in curative effect along.

In order to solve the main deficiencies of traditional Chinese medicine hypotensive, and enhance the traditional Chinese medicine hypotensive treatment effect, the applicant selects dozens of traditional Chinese medicinal materials which may have the effect of reducing blood pressure for further selections of better medicine effects of single-variety of medicinal materials and better extraction processes, and evaluates the optimization combinations of the extraction process. Finally, the alcohol-water dual-extraction of the Radix Paeoniae Alba is found, which hypotensive curative effect is far superior to that of conventional water extraction, and is better than the Western medicine Irbesartan, which hypotensive effect exceeds the conventional envisioned hypotensive effect. However, the hypotensive effect of the alcohol-water dual-extraction of the Radix Paeoniae Alba is relatively high only after the medicine is taken for 2 hours (quick-acting), which hypotensive effect in the quick-acting aspect is enhanced, but the curative effect of the Dendrobium Officinale powder application method is more prominent in the 24-hour hypotensive action (long-acting) (see China Patent Application No. 201710855728.6). The applications of both the alcohol-water dual-extraction of the Radix Paeoniae Alba and the Dendrobium Officinale powder are different from the Western medicine hypotensive drugs in terms of safety. They will not reduce the normal blood pressure, which is safer. The two single Chinese medicines of fast hypotensive effect (or the hypotensive peak effect) and the long hypotensive effect (or the hypotensive valley effect) respectively are matched and compatible for the research of various matching ratios, so that a synergistic effect is obtained in both aspects of a hypotensive “peak effect” (a hypotensive effect of 2 hours after administration) and a hypotensive “valley effect” (24-hour post-administration), and a hypotensive effect is comprehensively improved.

The preparation method of the present invention has the advantages that the preparation method can be used in cooperation, so that the effects of nourishing yin and suppressing yang, and nourishing blood and calming the liver are achieved. Besides, a proper preparation process is adopted, which greatly enhances its hypotensive effect, breaks through the conventional concept, and achieves the unexpected curative effect.

In the active ingredient of the Radix Paeoniae Alba, some are readily soluble in alcohol (such as: paeoniflorin, flavonoid aglycone, paeonol, carrot glycoside, volatile oil, benzoic acid, etc.), some are soluble in water (such as: paeoniflorin, flavones, saccharides, proteins, etc.), while some are both soluble (such as: paeoniflorin, tannins, etc.). The method disclosed by the present invention utilizes alcohol extraction for the powder of Radix Paeoniae Alba, so as to avoid the loss of the part of the components that is alcohol soluble only if the traditional method of water extraction is adopted. Besides, it also avoids insufficient extraction and small dissolution area occurring when alcohol extraction is directly applied to the Radix Paeoniae Alba drink. There is also a conventional method for directly orally taking Radix Paeoniae Alba powder, which hypotensive effect is still lower than the alcohol-water dual-extraction provided by the present invention. The main principle and reason may be that human body does not have a complete dissolve ability to alcohol extraction, which affects the absorption and utilization of the part of the components of the Radix Paeoniae Alba that is easily dissolved in the alcohol. In addition, the alcohol extract of the Radix Paeoniae Alba and water extract of the alcohol extracted Radix Paeoniae Alba have a synergistic effect in the hypotensive curative effect, which has been proven in the comparison between different hypotensive groups of the test: the hypotensive effect of the alcohol-water dual-extraction of Radix Paeoniae Alba group is significantly better than the water extraction of Radix Paeoniae Alba group, the alcohol extraction of Radix Paeoniae Alba group, and the Radix Paeoniae Alba powder group, while the hypotensive effects of the latter three groups are similar.

When the Dendrobium Officinale is taken directly after “grinding,” its pharmaceutical ingredients are more complete than utilizing traditional water extraction and the like. The drug gradually plays a role in the in-vivo digestion and absorption, which is that the traditional Chinese medicinal materials play a role of “slow release” for carrying out the hypotensive process in the body, so that the effect of reducing the blood pressure of 24 hours is more stable.

The alcohol-water dual-extraction of the Radix Paeoniae Alba is added with Dendrobium Officinale powder, which has high hypotensive effect for the hypertension patient, but, different from western hypotensive medicine, has no hypotensive effect to normal blood pressure, which is safer.

The present invention overcomes the traditional bias through compatibility optimization and extraction process optimization and enhances both the quick-acting and long-acting effects of traditional Chinese hypotensive medicine. The Radix Paeoniae Alba adopts the alcohol-water dual-extraction that combines the alcohol extraction and the water extraction method, which greatly enhances the quick-acting pressure reduction effect. It is then combined with the existing technology of utilizing Dendrobium Officinale powder (providing much better long-acting pressure reduction effect than water extraction). The effects of the synergy in the matching achieve the improvement in both quick-acting and long-acting pressure reductions. At the same time, the Dendrobium Officinale has the effects of nourishing yin, clearing heat, while the Radix Paeoniae Alba has the effects of nourishing blood and softening liver, and calming liver's yang. The coordination of these two traditional Chinese medicines helps to nourishing yin and reducing yang, nourishing blood and calming the liver, which greatly enhances the hypotensive effect, breaks through the conventional concept, and achieves the prevalence effect. In this way, the traditional Chinese medicine for reducing the blood pressure can possibly be introduced into the clinical practical application.

At the same time, it has also been discovered that the hypotensive action mechanism of the present formula is associated with a Type 1 receptor (ATIR) mRNA that significantly reduces angiotensin II, reduces the boosting action of angiotensin II on the receptor-generating level, and achieves hypotensive effect.

Compared with the prior art, the present invention has the following beneficial effects:

1. The extract of the alcohol-water dual-extraction of Radix Paeoniae Alba with the Dendrobium Officinale powder has outstanding curative effect on the treatment of hypertension. The hypotensive effect of the alcohol-water dual-extraction of Radix Paeoniae Alba group is significantly better than the water extraction of Radix Paeoniae Alba group, the alcohol extraction of Radix Paeoniae Alba group, and the Radix Paeoniae Alba powder group, while the hypotensive effects of the latter three groups are similar. The speed of recent blood pressure reduction after taking the extract of the alcohol-water dual-extraction of Radix Paeoniae Alba is significantly accelerated, and the reduced amplitude is significantly increased. Through compatibility of the Dendrobium Officinale powder application method (compared with the water extraction method, the long-acting pressure reduction effect is greatly improved), which effects of synergistic improvement of quick-acting and long-acting pressure reduction are achieved. At the same time, the Dendrobium Officinale has the effects of nourishing yin, clearing heat, while the Radix Paeoniae Alba has the effects of nourishing blood and softening liver, and calming liver's yang. The coordination of these two traditional Chinese medicines greatly enhances the hypotensive effect and achieves the prevalence effect. Its hypotensive effect can exceed the Western medicine Irbesartan. In this way, the traditional Chinese medicine for reducing the blood pressure can possibly be introduced into the clinical practical application.

2. Alcohol-water dual-extraction of Radix Paeoniae Alba (alcohol extraction+water extraction) reduces component loss and enhances performance than conventional water extraction: all medicinal materials are ground and the extraction interface is increased; ethanol extraction is first carried out to ensure that the alcohol soluble substance is dissolved out under the condition of lower temperature; water extraction is carried out after alcohol extraction, so that the alcohol soluble substance is not there to be affected by the hot water temperature when water extraction is carried out, so that the curative effect can be improved; the alcohol soluble substance and the water soluble substance of Radix Paeoniae Alba are both utilized, and they may provide a synergistic effect to a better hypotensive effect.

3. Conventionally, it may directly orally take Radix Paeoniae Alba powder, which hypotensive effect is still lower than the alcohol-water dual-extraction of Radix Paeoniae Alba. The main principle and reason may be that human body does not have a complete dissolve ability to alcohol extraction, which affects the absorption and utilization of the part of the components of the Radix Paeoniae Alba that is easily dissolved in the alcohol. The method of the present invention promotes the full utilization of the Radix Paeoniae Alba oral treatment by human body, and unexpectedly improves the curative effect.

4. Person with normal blood pressure is not affected by this selective adjustment of the hypertension. That is, the blood pressure of high blood pressure patient is reduced, but the blood pressure of normal blood pressure person is not reduced. Besides, the hypertension will not be reduced to be lower than the normal blood pressure. This is also an advantage with respect to most Western antihypertensive medicines.

5. The present invention makes it clear that the hypotensive action mechanism is associated with a Type 1 receptor (ATIR) mRNA that significantly reduces angiotensin II, reduces the boosting action of angiotensin II on the receptor-generating level, and achieves hypotensive effect.

### DETAILED DESCRIPTION OF THE PREFERRED EMBODIMENT

The following disclosure is to illustrate implementations of the present invention through specific embodiments.

### Embodiment 1

Eight servings of Dendrobium Officinale and six servings of Radix Paeoniae Alba:
taking 400 g Dendrobium Officinale and 300 g of Radix Paeoniae Alba that meet the standard of the Chinese Pharmacopoeia (2015);
grinding Radix Paeoniae Alba into powder, adding 7 times of total raw medicine amount of 70% ethanol, carrying out reflux extraction for 1 hour, extracting for 2 times, and water extracting the residue after the alcohol extractions;
water extracting for 2 times for the Radix Paeoniae Alba residue after the alcohol extractions: first water extraction: adding 10 times of total raw medicine amount of water, extracting for 1 hour at the temperature of 100° C.; and performing second water extraction: adding water at 10 times that of the raw medicine amount, and extracting for 1 hour at the temperature of 100° C.; combining and concentrating the water extracts; combining the alcohol extracts and water extracts of the Radix Paeoniae Alba; and
grinding the Dendrobium Officinale into powder, adding the powder into the combined extracting solution of the Radix Paeoniae Alba, adding appropriate auxiliary materials according to the conventional pharmaceutically rules, so as to finish preparing the Radix Paeoniae Alba and Dendrobium Officinale tablet. This is the sample of Embodiment 1. For oral administration, the daily dosage of an adult is equivalent to 14 g of the raw drug amount (which may be reduced to 3.5 g of the raw drug amount, and increased to 35 g of the raw drug amount).

### Embodiment 2

Eight servings of Dendrobium Officinale and two servings of Radix Paeoniae Alba:
taking 400 g Dendrobium Officinale and 100 g of Radix Paeoniae Alba that meet the standard of the Chinese Pharmacopoeia (2015);
grinding Radix Paeoniae Alba into powder, adding 7 times of total raw medicine amount of 70% ethanol, carrying out reflux extraction for 1 hour, extracting for 2 times, and water extracting the residue after the alcohol extractions;
water extracting for 2 times for the Radix Paeoniae Alba residue after the alcohol extractions: first water extraction: adding 10 times of total raw medicine amount of water, extracting for 1 hour at the temperature of 100° C.; and performing second water extraction: adding water at 10 times that of the raw medicine amount, and extracting for 1 hour at the temperature of 100° C.; combining and concentrating the water extracts; combining the alcohol extracts and water extracts of the Radix Paeoniae Alba; and
grinding the Dendrobium Officinale into powder, adding the powder into the combined extracting solution of the Radix Paeoniae Alba, adding appropriate auxiliary materials according to the conventional pharmaceutically rules, so as to finish preparing the Radix Paeoniae Alba and Dendrobium Officinale tablet. This is the sample of Embodiment 2. For oral administration, the daily dosage of an adult is equivalent to 10 g of the raw drug amount (which may be reduced to 2.5 g of the raw drug amount, and increased to 40 g of the raw drug amount).

### Embodiment 3

Eight servings of Dendrobium Officinale and 32 servings of Radix Paeoniae Alba:
taking 100 g Dendrobium Officinale and 400 g of Radix Paeoniae Alba that meet the standard of the Chinese Pharmacopoeia (2015);
grinding Radix Paeoniae Alba into powder, adding 7 times of total raw medicine amount of 70% ethanol, carrying out reflux extraction for 1 hour, extracting for 2 times, and water extracting the residue after the alcohol extractions;
water extracting for 2 times for the Radix Paeoniae Alba residue after the alcohol extractions: first water extraction: adding 10 times of total raw medicine amount of water, extracting for 1 hour at the temperature of 100° C.; and performing second water extraction: adding water at 10 times that of the raw medicine amount, and extracting for 1 hour at the temperature of 100° C.; combining and concentrating the water extracts; combining the alcohol extracts and water extracts of the Radix Paeoniae Alba; and
grinding the Dendrobium Officinale into powder, adding the powder into the combined extracting solution of the Radix Paeoniae Alba, adding appropriate auxiliary materials according to the conventional pharmaceutically rules, so as to finish preparing the Radix Paeoniae Alba and Dendrobium Officinale tablet. This is the sample of Embodiment 3. For oral administration, the daily dosage of an adult is equivalent to 10 g of the raw drug amount (which may be reduced to 2.5 g of the raw drug amount, and increased to 40 g of the raw drug amount).

### Embodiment 4

Eight servings of Dendrobium Officinale and six servings of Radix Paeoniae Alba:
taking 400 g Dendrobium Officinale and 300 g of Radix Paeoniae Alba that meet the standard of the Chinese Pharmacopoeia (2015);
grinding Radix Paeoniae Alba into powder, adding 7 times of total raw medicine amount of 70% ethanol, carrying out leaching extraction for 24 hour, extracting for 2 times, and water extracting the residue after the alcohol extractions;
water extracting for 2 times for the Radix Paeoniae Alba residue after the alcohol extractions: first water extraction: adding 10 times of total raw medicine amount of water, extracting for 1 hour at the temperature of 100° C.; and performing second water extraction: adding water at 10 times that of the raw medicine amount, and extracting for 1 hour at the temperature of 100° C.; combining and concentrating the water extracts; combining the alcohol extracts and water extracts of the Radix Paeoniae Alba; and
grinding the Dendrobium Officinale into powder, adding the powder into the combined extracting solution of the Radix Paeoniae Alba, adding appropriate auxiliary materials according to the conventional pharmaceutically rules, so as to finish preparing the Radix Paeoniae Alba and Dendrobium Officinale suspension particles. This is the sample of Embodiment 4. For oral administration, the daily dosage of an adult is equivalent to 7 g of the raw drug amount (which may be reduced to 3.5 g of the raw drug amount, and increased to 35 g of the raw drug amount).

### Embodiment 5

Eight servings of Dendrobium Officinale and six servings of Radix Paeoniae Alba:
taking 400 g Dendrobium Officinale and 300 g of Radix Paeoniae Alba that meet the standard of the Chinese Pharmacopoeia (2015);
grinding Radix Paeoniae Alba into powder, adding 7 times of total raw medicine amount of 70% ethanol, carrying out reflux extraction for 1 hour, extracting for 2 times, and water extracting the residue after the alcohol extractions;
gradient temperature water extracting for 2 times for the Radix Paeoniae Alba residue after the alcohol extractions: first water extraction: adding 10 times of total raw medicine amount of water, extracting for 1 hour at the temperature of 100° C.; and performing second water extraction: adding water at 10 times that of the raw medicine amount, and extracting for 1 hour at the temperature of 121° C.; combining and concentrating the water extracts; combining the alcohol extracts and water extracts of the Radix Paeoniae Alba; and
grinding the Dendrobium Officinale into powder, adding the powder into the combined extracting solution of the Radix Paeoniae Alba, adding appropriate auxiliary materials according to the conventional pharmaceutically rules, so as to finish preparing the Radix Paeoniae Alba and Dendrobium Officinale suspension particles. This is the sample of Embodiment 5. For oral administration, the daily dosage of an adult is equivalent to 7 g of the raw drug amount (which may be reduced to 3.5 g of the raw drug amount, and increased to 35 g of the raw drug amount).

### Embodiment 6

Eight servings of Dendrobium Officinale and six servings of Radix Paeoniae Alba:
taking 400 g Dendrobium Officinale and 300 g of Radix Paeoniae Alba that meet the standard of the Chinese Pharmacopoeia (2015);
grinding Radix Paeoniae Alba into powder, adding 7 times of total raw medicine amount of 70% ethanol, carrying out leaching extraction for 24 hour, extracting for 2 times, and water extracting the residue after the alcohol extractions;
gradient temperature water extracting for 2 times for the Radix Paeoniae Alba residue after the alcohol extractions: first water extraction: adding 10 times of total raw medicine amount of water, extracting for 1 hour at the temperature of 100° C.; and performing second water extraction: adding water at 10 times that of the raw medicine amount, and extracting for 1 hour at the temperature of 121° C.; combining and concentrating the water extracts; combining the alcohol extracts and water extracts of the Radix Paeoniae Alba; and
grinding the Dendrobium Officinale into powder, adding the powder into the combined extracting solution of the Radix Paeoniae Alba, adding appropriate auxiliary materials according to the conventional pharmaceutically rules, so as to finish preparing the Radix Paeoniae Alba and Dendrobium Officinale suspension particles. This is the sample of Embodiment 6. For oral administration, the daily dosage of an adult is equivalent to 7 g of the raw drug amount (which may be reduced to 3.5 g of the raw drug amount, and increased to 35 g of the raw drug amount).

### Experiment 1

### Experimental Study of Hypotensive Effect on Hereditary Hypertension Rat (SHR) by the Sample of the Alcohol-Water Dual-Extraction of Radix Paeoniae Alba

### Material

Rats: old age (over 48 weeks old) hereditary hypertension rats (SHR) and the normal controls were Wistar rats.

The sample of the alcohol-water dual-extraction of Radix Paeoniae Alba: taking 500 g of the Radix Paeoniae Alba that meet the standards of the Chinese Pharmacopoeia (2015), grinding into powder, adding 7 times of total raw medicine amount of 70% ethanol, carrying out reflux extraction for 1 hour, extracting for 2 times, and water extracting the residue for 2 times after the alcohol extractions. The alcohol extracts and the water extracts are respectively concentrated and combined. Appropriate auxiliary materials are added according to the conventional pharmaceutically rules. The prepared medicine is ready for later use.

The sample of the alcohol extracted Radix Paeoniae Alba: taking 500 g of the Radix Paeoniae Alba that meet the standards of the Chinese Pharmacopoeia (2015), grinding into powder, adding 7 times of total raw medicine amount of ethanol, carrying out reflux extraction for 1 hour extracting for 2 times. The alcohol extract is concentrated for later use.

The sample of the water extracted Radix Paeoniae Alba: taking 500 g of the Radix Paeoniae Alba that meet the standards of the Chinese Pharmacopoeia (2015), grinding into powder, water extracting for 2 times: first water extraction: adding 10 times of total raw medicine amount of water, extracting for 1 hour at the temperature of 100° C.; and second water extraction: adding water at 10 times of the raw medicine amount, and extracting for 1 hour at the temperature of 100° C. The extracts are combined and concentrated for later use.

The Radix Paeoniae Alba powder: taking 500 g of the Radix Paeoniae Alba that meet the standards of the Chinese Pharmacopoeia (2015), and grinding into powder.

Western hypotensive medicine for the control: Irbesartan tablet (Aprovel®), 150 mg/tablet, Sanofi Inc.

Rat blood pressure measurement: BP-98 A type non-invasive tail arterial blood pressure measuring instrument, Softron Co., Ltd. (Japan).

### Method

Grouping treatment: All rats were adaptively fed for a week, measured with blood pressure and weight, and set into groups. SHR rats were divided into alcohol-water dual-extraction of Radix Paeoniae Alba large dosage group for the dosage of 3.2 g/kg body weight, alcohol-water dual-extraction of Radix Paeoniae Alba medium dosage group for the dosage of 0.8 g/kg body weight, alcohol-water dual-extraction of Radix Paeoniae Alba small dosage group for the dosage of 0.2 g/kg body weight, alcohol extraction of Radix Paeoniae Alba group for the dosage of 0.8 g/kg body weight, water extraction of Radix Paeoniae Alba group for the dosage of 0.8 g/kg body weight, Radix Paeoniae Alba powder group for the dosage of 0.8 g/kg body weight; utilizing gastric infusion; hypertension model control group receives equal amount of physiological saline; Irbesartan control group receives 15 mg/Kg body weight of Irbesartan; and normal control group receives equal amount of physiological saline. Each group has 8 rats. The treatment last for 3 weeks (multiple of 3-12 weeks of treatments are performed, the results can be repeated, and typical experimental results are reported below).

Blood pressure measurement: The Rats were pre-heated to 38° C. for 5-10 mins to avoid less blood flow of the tail that affects the pressure measurement. Three measurements are continuously carried out as the rat was in an awake and quiet state. The average value of the 3 measurements was considered as the blood pressure of the rat. The blood pressure is measured prior to the treatment, after the first administration, and 2 hours after each administration every week.

### Results

Blood pressure data (x̅±S): See Table 1 and 2 of Experiment 1. When treated for 3 weeks, the blood pressure of the alcohol-water dual-extraction of Radix Paeoniae Alba large dosage group was 166.7±3.71/140.6±2.71 mmHg, the blood pressure of the medium dosage group was 173.6±0.32/145.8±2.34 mmHg, and the blood pressure of the small dosage group was 189.3±3.85/153.9±2.95 mmHg, which were all significantly lower than the blood pressure of the hypertension model control group of 216.8±2.69/165.8±5.79 mmHg (P<0.01).

The dosage for the alcohol-water dual-extraction of Radix Paeoniae Alba medium dosage group is 0.8 g/kg body weight, which is the same with the dosages for the alcohol extraction of Radix Paeoniae Alba group, the water extraction of Radix Paeoniae Alba group, and the Radix Paeoniae Alba powder group. Therefore, the hypotensive curative effect is comparable. When treated for 3 weeks, the blood pressure of the water extraction of Radix Paeoniae Alba group is 181.7±0.98/152.4±1.99 mmHg, the blood pressure of the alcohol extraction of Radix Paeoniae Alba group is 181.5±1.11/151.2±1.93 mmHg, and the blood pressure of the Radix Paeoniae Alba powder group is 181.0±1.78/150.3±1.95 mmHg, which are all higher than it of the alcohol-water dual-extraction of Radix Paeoniae Alba medium dosage group (P<0.01 or P<0.05).

The research results showed that the hypotensive curative effect of the alcohol-water dual-extraction of Radix Paeoniae Alba is significantly better than that of traditional water extraction, and is also better than that of Radix Paeoniae Alba alcohol extraction as well as the Radix Paeoniae Alba powder. The alcohol extract and the water extract in the alcohol-water dual-extraction of Radix Paeoniae Alba have a synergistic effect of increasing the curative effect. The hypotensive curative effect of the Radix Paeoniae Alba powder is also lower than the sample of the alcohol-water dual-extraction of Radix Paeoniae Alba. It is possibly that when the Radix Paeoniae Alba powder is orally taken directly, human body does not have a complete dissolve ability to alcohol extraction, which affects the absorption and utilization of the part of the components of the Radix Paeoniae Alba that is easily dissolved in the alcohol.

The hypotensive curative effects of the alcohol-water dual-extraction of Radix Paeoniae Alba large dosage group (3.2 g of raw drug/Kg body weight) and medium dosage group (0.8 g of raw drug/Kg body weight) are both better than it of the Irbesartan control group (P<0.01 or P<0.05).

The dosages used in this experiment for rats corresponding to the equivalent dosages for adults:
The equivalent dosage for adults corresponding to the alcohol-water dual-extraction of Radix Paeoniae Alba large dosage group is about 32 g of raw Radix Paeoniae Alba/day;
The equivalent dosage for adults corresponding to the alcohol-water dual-extraction of Radix Paeoniae Alba medium dosage group is about 8 g of raw Radix Paeoniae Alba/day;
The equivalent dosage for adults corresponding to the alcohol-water dual-extraction of Radix Paeoniae Alba small dosage group is about 2 g of raw Radix Paeoniae Alba/day;
The equivalent dosage for adults corresponding to the Irbesartan control group is about 150 mg of Irbesartan/day; and
The equivalent dosages for adults corresponding to the alcohol extraction of Radix Paeoniae Alba group, the water extraction of Radix Paeoniae Alba group, and the Radix Paeoniae Alba powder group are the same, which is about 8 g of raw Radix Paeoniae Alba/day (equal to the raw medicine amount of the alcohol-water dual-extraction of Radix Paeoniae Alba medium dosage group).

### Conclusion

All of the large dosage (3.2 g of raw medicine/kg body weight), medium dosage (0.8 g of raw medicine/kg body weight), and small dosage (0.2 g of raw medicine/kg body weight) are able to provide hypotensive effect to the SHR. The overall hypotensive curative effects of the large dosage (3.2 g of raw medicine/kg body weight) and the medium dosage (0.8 g of raw medicine/kg body weight) are better than it of the Irbesartan (15 mg/kg body weight).

The research results also showed that the hypotensive curative effect of the sample of the alcohol-water dual-extraction of Radix Paeoniae Alba is significantly better than that of traditional water extraction, and is also better than that of Radix Paeoniae Alba alcohol extraction as well as the Radix Paeoniae Alba powder.

Experiment 1: Table 1. The effect of the sample of the alcohol-water dual-extraction of Radix Paeoniae Alba on the systolic pressure of SHR rats (x̅±S, n=8, unit: mmHg)

| | Normal | Irbesartan | Dual-extraction | Dual-extraction |
|---|---|---|---|---|
| | Control Group | Control Group | Large Dosage Group | Medium Dosage Group |
| Before Treatment | 131.5 ± 4.66^{▴} | 215.2 ± 4.45 | 215.7 ± 4.77 | 216.5 ± 4.87 |
| First Treated | 133.2 ± 3.48^{▴} | 181.6 ± 3.12^{▴} | 170.5 ± 1.45^{▴▪} | 175.0 ± 1.21^{▴□} |
| Treated for 1 Week | 133.7 ± 4.23^{▴} | 180.4 ± 3.56^{▴} | 168.4 ± 1.57^{▴▪} | 174.3 ± 2.74^{▴□} |
| Treated for 2 Weeks | 135.3 ± 3.21^{▴} | 178.6 ± 2.04^{▴} | 167.7 ± 2.64^{▴▪} | 173.7 ± 1.47^{▴□} |
| Treated for 3 Weeks | 137.1 ± 4.35^{▴} | 177.8 ± 2.12^{▴} | 166.7 ± 3.71^{▴▪} | 173.6 ± 0.32^{▴□} |

| | Dual-extraction | Alcohol | Water | |
|---|---|---|---|---|
| Model Group | Small Dosage Group | Extraction Group | Extraction Group | Powder Group |
| 216.4 ± 3.75 | 215.9 ± 3.08 | 215.7 ± 2.85 | 216.6 ± 3.68 | 216.6 ± 3.52 |
| 216.5 ± 2.86 | 191.3 ± 3.79^{▴} | 181.4 ± 1.64^{▴★} | 182.5 ± 1.18^{▴★} | 182.1 ± 0.69^{▴★} |
| 214.8 ± 3.24 | 188.8 ± 3.15^{▴} | 182.3 ± 1.03^{▴★} | 181.3 ± 3.20^{▴⋆} | 180.5 ± 1.58^{▴⋆} |
| 215.5 ± 4.45 | 188.7 ± 2.72^{▴} | 182.2 ± 1.60^{▴★} | 181.0 ± 1.36^{▴★} | 180.3 ± 1.34^{▴★} |
| 216.8 ± 3.69 | 189.3 ± 3.85^{▴} | 181.5 ± 1.11^{▴★} | 181.7 ± 0.98^{▴★} | 181.0 ± 1.78^{▴⋆} |

note:
for the groups compared with the model group, ΔP < 0.05, ^{▴}P < 0.01; for the large dosage group and medium dosage group compared with the Irbesartan control group, ^{□}P < 0.05, ^{▪}P < 0.01; and for the alcohol extraction group, water extraction group, and powder group compared with the medium dosage group, ^{⋆}P < 0.05, ^{★}P < 0.01.

Experiment 1: Table 2. The effect of the sample of the alcohol-water dual-extraction of Radix Paeoniae Alba on the diastolic pressure of SHR rats (x̅±S, n=8, unit: mmHg)

| | Normal | Irbesartan | Dual-extraction | Dual-extraction |
|---|---|---|---|---|
| | Control Group | Control Group | Large Dosage Group | Medium Dosage Group |
| Before Treatment | 99.6 ± 5.33 | 166.7 ± 4.21 | 166.5 ± 4.66 | 165.8 ± 1.75 |
| First Treated | 98.2 ± 4.73^{▴} | 155.2 ± 3.25^{▴} | 145.0 ± 3.45^{▴▪} | 149.6 ± 1.37^{▴□} |
| Treated for 1 Week | 97.6 ± 4.89^{▴} | 150.8 ± 2.89^{▴} | 143.4 ± 1.58^{▴□} | 148.9 ± 0.96^{▴} |
| Treated for 2 Weeks | 98.7 ± 5.69^{▴} | 151.8 ± 2.71^{▴} | 141.7 ± 2.64^{▴▪} | 145.9 ± 2.74^{▴□} |
| Treated for 3 Weeks | 99.2 ± 6.83^{▴} | 152.9 ± 2.89^{▴} | 140.6 ± 2.71^{▴▪} | 145.8 ± 2.34^{▴□} |

| | Dual-extraction | Alcohol | Water | |
|---|---|---|---|---|
| Model Group | Small Dosage Group | Extraction Group | Extraction Group | Powder Group |
| 166.5 ± 3.62 | 165.4 ± 3.01 | 165.2 ± 2.01 | 165.2 ± 3.58 | 165.5 ± 3.46 |
| 165.3 ± 3.68 | 149.2 ± 2.81^{▴} | 148.6 ± 2.04^{▴} | 149.6 ± 1.85^{▴} | 148.8 ± 2.03^{▴} |
| 165.4 ± 4.18 | 150.8 ± 3.70^{▴} | 149.1 ± 2.18^{▴} | 149.5 ± 1.75^{▴} | 150.0 ± 1.70^{▴} |
| 165.5 ± 4.01 | 151.3 ± 3.15^{▴} | 150.0 ± 1.31^{▴⋆} | 152.3 ± 2.63^{▴⋆} | 151.4 ± 1.20^{▴⋆} |
| 165.8 ± 5.79 | 153.9 ± 2.95^{▴} | 151.2 ± 1.93^{▴⋆} | 152.4 ± 1.99^{▴⋆} | 150.3 ± 1.95^{▴⋆} |

note:
for the groups compared with the model group, ΔP < 0.05, ^{▴}P < 0.01; for the large dosage group and medium dosage group compared with the Irbesartan control group, ^{□}P < 0.05, ^{▪}P < 0.01; and for the alcohol extraction group, water extraction group, and powder group compared with the medium dosage group, ^{⋆}P < 0.05, ★P < 0.01.

### Experiment 2

### Experimental Study of Hypotensive Effect on Hereditary Hypertension Rat (SHR) by the Sample of the Embodiment 1

### Material

Rats: old age (over 48 weeks old) hereditary hypertension rats (SHR) and the normal controls were Wistar rats.

Chinese medicine of the experiment: the sample of the Embodiment 1 (alcohol-water dual-extraction of Radix Paeoniae Alba with Dendrobium Officinale powder added), dissolved with hot water before use.

The sample of the alcohol-water dual-extraction of Radix Paeoniae Alba: taking 500 g of the Radix Paeoniae Alba that meet the standards of the Chinese Pharmacopoeia (2015), grinding into powder, adding 7 times of total raw medicine amount of 70% ethanol, carrying out reflux extraction for 1 hour, extracting for 2 times, and water extracting the residue for 2 times after the alcohol extractions. The alcohol extracts and the water extracts are respectively concentrated and combined. The prepared medicine is ready for later use.

The Dendrobium Officinale powder: taking 500 g of the Dendrobium Officinale that meet the standards of the Chinese Pharmacopoeia (2015), and grinding into powder.

Western hypotensive medicine for the control: Irbesartan tablet (Aprovel®), 150 mg/tablet, Sanofi Inc.

Rat blood pressure measurement: BP-98 A type non-invasive tail arterial blood pressure measuring instrument, Softron Co., Ltd. (Japan).

### Method

1. Grouping treatment: All rats were adaptively fed for a week, measured with blood pressure and weight, and set into groups. SHR rats were grouped: sample of the Embodiment 1 (alcohol-water dual-extraction of Radix Paeoniae Alba with Dendrobium Officinale powder added)-large dosage group for the dosage of 3.5 g/kg body weight, sample of the Embodiment 1-medium dosage group for the dosage of 1.4 g/kg body weight, sample of the Embodiment 1-small dosage group for the dosage of 0.35 g/kg body weight, Dendrobium Officinale powder for the dosage of raw medicine of 0.8 g/kg body weight (which is the same as the raw medicine amount of the Dendrobium Officinale powder (0.8 g/kg body weight) contained in 1.4 g/Kg body weight of the sample of the Embodiment 1), alcohol-water dual-extraction of Radix Paeoniae Alba for the dosage of raw medicine of 0.6 g/kg body weight (which is the same as the raw medicine amount of the Radix Paeoniae Alba (0.6 g/kg body weight) contained in 1.4 g/Kg body weight of the sample of the Embodiment 1); utilizing gastric infusion; hypertension model control group receives equal amount of physiological saline; Irbesartan control group receives 15 mg/Kg body weight of Irbesartan; and normal control group receives equal amount of physiological saline. Each group has 8 rats. The treatment last for 3 weeks (multiple of 3-12 weeks of treatments are performed, the results can be repeated, and typical experimental results are reported below).

2. Blood pressure measurement: The Rats were pre-heated to 38° C. for 5-10 mins to avoid less blood flow of the tail that affects the pressure measurement. Three measurements are continuously carried out as the rat was in an awake and quiet state. The average value of the 3 measurements was considered as the blood pressure of the rat. The blood pressure is measured prior to the treatment, after the first administration, and 2 hours and 24 hours after each administration every week.

3. Expression level detection of mRNA in tissue:

After 3 weeks of treatment, the rat of the normal group, the model group, the sample of the Embodiment 1-medium dosage group, and the Dendrobium Officinale powder group are anaesthetized and executed, and then the kidney tissues are taken, put into liquid nitrogen for rapid freezing, and stored at −80° C. The kidney tissues are for the subsequent measurement of ATIR mRNA expression level. It used qPCR, and the relative expression amount of mRNA of target gene, the type 1 receptor of angiotensin II, to the relative expression of the reference gene beta-ACTIN is calculated.

Total RNA extraction: extraction reagent RNAisoplus9109, and the steps: (1) taking 80-100 mg of tissue, adding liquid nitrogen, grinding for 4-5 times into powder, adding 1 ml Trizol to continue grinding to a transparent state, and transferring to a 1.5 ml centrifuge tube to stand at room temperature (4-25° C.) for 5 min. (2) adding 0.2 mL of trichloromethane, fully mixing evenly in emulsion, standing at room temperature for 5 min; centrifuging at 12,000×g for 15 min at 4° C. (3) sucking 400 μL of supernatant to a new 1.5 mL tube, adding 600-800 μL of isopropanol, uniformly mixing, standing at room temperature for 10 min, centrifuging at 12,000×g for 10 min at 4° C. (4) discarding the supernatant, adding 1 mL of 75% ethanol (prepared with DEPC water) for washing and precipitating, standing for 3 min at room temperature, and centrifuging at 12,000×g for 10 min at 4° C. (5) discarding the supernatant as much as possible, drying at room temperature (no heating) for 5-10 min. The sample is dissolved with an appropriate amount of DEPC water, and its concentration and OD value are measured.

RT-PCR: reagent: PrimeScript™ RT Master Mix (Perfect Real Time) RR036A, and the steps are as follows:

### (1) Preparing the Mixture in the Tube (RNase Free):

template RNA 500 ng
5×PrimeScript RT Master Mix 2 μL
RNase free ddH2O Up to 10 μL

### (2) Mixing the Mixture Evenly; the Reaction Conditions: 37° C., 15 Mins (RT Reaction) at 85° C., 5 s at 4° C., ∞.

diluting the cDNA product for 5 times for qPCR.
qPCR reaction system:
SYBR Premix Ex Taq II 10 μL
Primer R (10 μM) 0.8 μL
Primer F (10 μM) 0.8 μL
ROX Reference Dye 10.4 μL
template cDNA 2 μL
sterilized distilled water 6 μL
Reaction conditions of qPCR: 95° C., 30 s, 95° C., 5 s, 60° C., 30 s, for 40 cycles; 95° C., 15 s, 60° C., 1 min, 95° C., 15 s, for the dissociation curve.

### Sequences of the Primers of the Gene

| | | | Product |
|---|---|---|---|
| Gene | Upstream Primer | Downstream Primer | (bp) |
| AT1R | 5′-GCTTCAACCTCTACGCCAGTGTG-3′ | 5′-CAGCCAGATGATGATGCAGGTGAC-3′ | 137 |
| β-actin | 5′-GGAGATTACTGCCCTGGCTCCTA-3′ | 5′-GACTCATCGTACTCCTGCCTGCTG-3′ | 150 |

### Results

1. Two Hours after Administration, Blood Pressure Data (x̅±S): See Table 1 and 2 of Experiment 2.

When treated for 3 weeks, the blood pressure of the sample of the Embodiment 1 (alcohol-water dual-extraction of Radix Paeoniae Alba with Dendrobium Officinale powder added)-large dosage group was 167.8±2.61/136.1±4.26 mmHg, the blood pressure of the medium dosage group was 170.1±1.69/139.6±2.32 mmHg, and the blood pressure of the small dosage group was 190.4±3.68/152.3±3.76 mmHg, which were all significantly lower than the blood pressure of the hypertension model control group of 217.4±5.68/167.4±4.21 mmHg (P<0.01). Here the blood pressures of the large dosage group and the medium dosage group are both significantly lower than it of the Irbesartan control group of 178.9±3.87/149.5±3.24 mmHg (P<0.01 or P<0.05), which indicates that the extents of the fast hypotensive effect of two hours of administration of the large dosage group and the medium dosage group both exceed it of the Western medicine Irbesartan.

The raw medicine of the Dendrobium Officinale for the Embodiment 1 (alcohol-water dual-extraction of Radix Paeoniae Alba with Dendrobium Officinale powder added)-medium dosage group is 0.8 g/kg body weight, which is the same as the raw medicine of the Dendrobium Officinale for the Dendrobium Officinale powder group and the raw medicine of the Radix Paeoniae Alba for the Embodiment 1 (alcohol-water dual-extraction of Radix Paeoniae Alba with Dendrobium Officinale powder added)-medium dosage group is 0.6 g/kg body weight, which is the same as the raw medicine of Radix Paeoniae Alba of the alcohol-water dual-extraction group. Therefore, the blood pressures of these three groups are comparable. After 3 weeks of treating, the blood pressure of the Dendrobium Officinale powder group is 182.5±2.98/151.8±2.81 mmHg and the blood pressure of the alcohol-water dual-extraction group is 175.6±1.75/145.9±2.53 mmHg. Both are significantly higher than the Embodiment 1 (alcohol-water dual-extraction of Radix Paeoniae Alba with Dendrobium Officinale powder added)-medium dosage group (P<0.01 or P<0.05). This indicates that the matching and combine use of the alcohol-water dual-extraction of Radix Paeoniae Alba and the Dendrobium Officinale powder has a synergistic effect in enhancing the fast hypotensive curative effect (2 hours after administration).

2. Two Hours after Administration, Blood Pressure Data (x̅±S): See Table 1 and 24 of Experiment 2.

When treated for 3 weeks, the blood pressure of the sample of the Embodiment 1 (alcohol-water dual-extraction of Radix Paeoniae Alba with Dendrobium Officinale powder added)-large dosage group was 187.3±3.18/143.1±3.21 mmHg, the blood pressure of the medium dosage group was 193.5±1.55/145.3±2.20 mmHg, and the blood pressure of the small dosage group was 204.8±5.76/156.6±4.05 mmHg, which were all significantly lower than the blood pressure of the hypertension model control group of 216.3±3.57/166.4±3.99 mmHg (P<0.01). Here the blood pressures of the large dosage group and the medium dosage group are both significantly lower than it of the Irbesartan control group of 198.7±2.24/154.1±2.08 mmHg (P<0.01 or P<0.05), which indicates that the extents of the long hypotensive effect of 24 hours of administration of the large dosage group and the medium dosage group both exceed it of the Western medicine Irbesartan.

The raw medicine of the Dendrobium Officinale for the Embodiment 1 (alcohol-water dual-extraction of Radix Paeoniae Alba with Dendrobium Officinale powder added)-medium dosage group is 0.8 g/kg body weight, which is the same as the raw medicine of the Dendrobium Officinale for the Dendrobium Officinale powder group and the raw medicine of the Radix Paeoniae Alba for the Embodiment 1 (alcohol-water dual-extraction of Radix Paeoniae Alba with Dendrobium Officinale powder added)-medium dosage group is 0.6 g/kg body weight, which is the same as the raw medicine of Radix Paeoniae Alba of the alcohol-water dual-extraction group. Therefore, the blood pressures of these three groups are comparable. After 3 weeks of treating, the blood pressure of the Dendrobium Officinale powder group is 199.2±1.96/152.9±3.61 mmHg and the blood pressure of the alcohol-water dual-extraction group is 203.8±2.11/154.9±3.86 mmHg. Both are significantly higher than the Embodiment 1 (alcohol-water dual-extraction of Radix Paeoniae Alba with Dendrobium Officinale powder added)-medium dosage group (P<0.01 or P<0.05). This indicates that the matching and combine use of the alcohol-water dual-extraction of Radix Paeoniae Alba and the Dendrobium Officinale powder also has a synergistic effect in enhancing the long hypotensive curative effect (24 hours after administration).

The results indicates that the hypotensive curative effect of the Embodiment 1 sample of the alcohol-water dual-extraction of Radix Paeoniae Alba with Dendrobium Officinale powder added is significantly better than all of it of the alcohol-water dual-extraction of Radix Paeoniae Alba solely, it of the Dendrobium Officinale powder solely, and it of the Western medicine Irbesartan. The matching and blending of the fast-acting advantage of the alcohol-water dual-extraction of the Radix Paeoniae Alba and the long lasting advantage of the Dendrobium Officinale powder enhances the curative effect coordinately.

3. The Relative Expression Amount (x̅±S) of the mRNA of the Type 1 Receptor of Angiotensin II: See Table 3 of Experiment 2.

The relative expression amounts of the mRNA of ATIR of the samples of the Embodiment 1 (alcohol-water dual-extraction of Radix Paeoniae Alba with Dendrobium Officinale powder added)-medium dosage group and the Dendrobium Officinale powder group are significantly lower than it of the model group (P<0.01). The relative expression amount of the mRNA of ATIR of the model group is significantly higher than it of the normal group (P<0.05).

Experiment 2: Table 3. The Relative Expression Amount of the mRNA of the Type 1 Receptor of Angiotensin II (AT1R)

| | AGT1R mRNA | P value comparing |
|---|---|---|
| Group (n = 8) | expression (x̅ ± S) | to the model group |
| Dendrobium Officinale | 0.00788 ± 0.00035 | <0.01 |
| Powder Group | | |
| Embodiment 1 Sample- | 0.00622 ± 0.00209 | <0.01 |
| Medium Dosage Group | | |
| Model Group | 0.01616 ± 0.00094 | |
| Normal Control Group | 0.01312 ± 0.00246 | <0.05 |

The dosages used in this experiment for rats corresponding to the equivalent dosages for adults:
the equivalent dosage for adults corresponding to the Embodiment 1 sample (alcohol-water dual-extraction of Radix Paeoniae Alba with Dendrobium Officinale powder added)-large dosage group is about 20 g of Dendrobium Officinale, 15 g of Radix Paeoniae Alba, which is totally 35 g of raw medicine/day;
the equivalent dosage for adults corresponding to the Embodiment 1 sample (alcohol-water dual-extraction of Radix Paeoniae Alba with Dendrobium Officinale powder added)-medium dosage group is about 8 g of Dendrobium Officinale, 6 g of Radix Paeoniae Alba, which is totally 14 g of raw medicine/day;
the equivalent dosage for adults corresponding to the Embodiment 1 sample (alcohol-water dual-extraction of Radix Paeoniae Alba with Dendrobium Officinale powder added)-small dosage group is about 2 g of Dendrobium Officinale, 1.5 g of Radix Paeoniae Alba, which is totally 3.5 g of raw medicine/day;
the equivalent dosage for adults corresponding to the Irbesartan control group is about 150 mg of Irbesartan/day;
the equivalent dosage for adults corresponding to the sample of the alcohol-water dual-extraction of Radix Paeoniae Alba group is about 6 g of raw Radix Paeoniae Alba/day (equal to the raw medicine amount of the Radix Paeoniae Alba of the Embodiment 1 sample-medium dosage group); and
the equivalent dosage for adults corresponding to the Dendrobium Officinale powder group is about 8 g of raw Dendrobium Officinale/day (equal to the raw medicine amount of the Dendrobium Officinale of the Embodiment 1 sample-medium dosage group).

### Conclusion

All of the Embodiment 1 sample (alcohol-water dual-extraction of Radix Paeoniae Alba with Dendrobium Officinale powder added)-large dosage (3.5 g of raw medicine/kg body weight), medium dosage (1.4 g of raw medicine/kg body weight), and small dosage (0.35 g of raw medicine/kg body weight) are able to provide significant hypotensive effect to the SHR. The overall hypotensive curative effects of the large dosage (3.5 g of raw medicine/kg body weight) and the medium dosage (1.4 g of raw medicine/kg body weight) are better than it of the Irbesartan (15 mg/kg body weight).

The results indicates that the hypotensive curative effect of the Embodiment 1 sample of the alcohol-water dual-extraction of Radix Paeoniae Alba with Dendrobium Officinale powder added is significantly better than all of it of the alcohol-water dual-extraction of Radix Paeoniae Alba solely, it of the Dendrobium Officinale powder solely, and it of the Western medicine Irbesartan. The matching and blending of the fast-acting advantage of the alcohol-water dual-extraction of the Radix Paeoniae Alba and the long lasting advantage of the Dendrobium Officinale powder enhances the curative effect coordinately.

The hypotensive action mechanisms of the Embodiment 1 sample and the Dendrobium Officinale powder are associated with a Type 1 receptor (AT1R) mRNA that significantly reduces angiotensin II, reduces the boosting action of angiotensin II on the receptor-generating level, and achieves hypotensive effect.

Experiment 2: Table 1. The Effect of the Sample of the Embodiment 1 on the Systolic Pressure of SHR Rats (x̅±S, n=8, Unit: mmHg)

| | Normal Control Group | Irbesartan Control Group | Large Dosage Group | Medium Dosage Group |
|---|---|---|---|---|
| Before Treatment | 132.7 ± 5.82^{▴} | 215.3 ± 5.87 | 216.7 ± 5.76 | 215.6 ± 4.05 |
| Treat 1 time, 2 hrs | 133.2 ± 3.48^{▴} | 182.4 ± 3.89^{▴} | 166.3 ± 2.43^{▴▪} | 170.1 ± 3.01^{▴▪} |
| Treat 1 time, 24 hrs | 135.82 ± 5.72^{▴} | 198.7 ± 3.61^{▴} | 186.5 ± 3.35^{▴▪} | 197.8 ± 2.78^{▴} |
| Treat 1 week, 2 hrs | 136.4 ± 3.76^{▴} | 181.2 ± 3.34^{▴} | 169.6 ± 3.76^{▴▪} | 173.3 ± 2.85^{▴□} |
| Treat 1 week, 24 hrs | 134.7 ± 4.86^{▴} | 197.8 ± 2.87^{▴} | 187.3 ± 2.49^{▴▪} | 196.5 ± 1.67^{▴} |
| Treat 2 weeks, 2 hrs | 136.4 ± 5.14^{▴} | 177.5 ± 2.23^{▴} | 168.9 ± 2.53^{▴▪} | 171.9 ± 1.69^{▴□} |
| Treat 2 weeks, 24 hrs | 138.1 ± 4.38^{▴} | 199.9 ± 2.36^{▴} | 188.4 ± 3.24^{▴▪} | 194.8 ± 2.69^{▴□} |
| Treat 3 weeks, 2 hrs | 136.8 ± 5.21^{▴} | 178.9 ± 3.87^{▴} | 167.8 ± 2.61^{▴▪} | 170.1 ± 1.69^{▴□} |
| Treat 3 weeks, 24 hrs | 137.4 ± 4.59^{▴} | 198.7 ± 2.24^{▴} | 187.3 ± 3.18^{▴▪} | 193.5 ± 1.55^{▴□} |

| | | | Dendrobium Officinale Powder | |
|---|---|---|---|---|
| | Small Dosage Group | Dual-extraction Group | Group | Model Group |
| Before Treatment | 214.9 ± 4.38 | 216.8 ± 3.94 | 214.5 ± 4.65 | 215.7 ± 4.56 |
| Treat 1 time, 2 hrs | 189.2 ± 4.85^{▴} | 177.1 ± 1.41^{▴⋆} | 181.2 ± 1.25^{▴⋆} | 216.3 ± 3.89 |
| Treat 1 time, 24 hrs | 205.8 ± 3.72^{Δ} | 202.3 ± 1.31^{▴⋆} | 200.1 ± 3.82^{▴⋆} | 214.1 ± 4.67 |
| Treat 1 week, 2 hrs | 187.5 ± 5.37^{▴} | 175.3 ± 1.03^{▴} | 179.3 ± 3.56^{▴⋆} | 215.7 ± 3.25 |
| Treat 1 week, 24 hrs | 204.3 ± 4.87^{▴} | 201.6 ± 2.08^{▴⋆} | 201.2 ± 2.56^{▴⋆} | 215.3 ± 3.78 |
| Treat 2 weeks, 2 hrs | 189.9 ± 4.76^{▴} | 173.7 ± 1.48^{▴} | 179.9 ± 2.57^{▴★} | 216.7 ± 3.76 |
| Treat 2 weeks, 24 hrs | 205.5 ± 2.87^{▴} | 202.1 ± 2.78^{▴★} | 199.1 ± 2.34^{▴⋆} | 215.8 ± 4.35 |
| Treat 3 weeks, 2 hrs | 190.4 ± 3.68^{▴} | 175.6 ± 1.75^{▴⋆} | 182.5 ± 2.98^{▴★} | 217.4 ± 5.68 |
| Treat 3 weeks, 24 hrs | 204.8 ± 5.76^{▴} | 203.8 ± 2.11^{▴★} | 199.2 ± 1.96^{▴⋆} | 216.3 ± 3.57 |

for the groups compared with the model group, ^{Δ}P < 0.05, ^{▴}P < 0.01; for the large dosage group and medium dosage group compared with the Irbesartan control group, ^{□}P < 0.05, ^{▪}P < 0.01; and for the alcohol-water dual-extraction group and the Dendrobium Officinale powder group compared with the medium dosage group, ^{⋆}P < 0.05, ^{★}P < 0.01.

Experiment 2: Table 2. The Effect of the Sample of the Embodiment 1 on the Diastolic Pressure of SHR Rats (x̅±S, n=8, Unit: mmHg)

| | Normal Control Group | Irbesartan Control Group | Large Dosage Group | Medium Dosage Group |
|---|---|---|---|---|
| Before Treatment | 97.4 ± 6.52^{▴} | 166.7 ± 4.21 | 166.2 ± 5.76 | 162.5 ± 5.25 |
| Treat 1 time, 2 hrs | 96.8 ± 5.64^{▴} | 150.1 ± 3.25^{▴} | 142.1 ± 1.45^{▴▪} | 146.8 ± 2.60^{▴} |
| Treat 1 time, 24 hrs | 96.4 ± 3.76^{▴} | 156.6 ± 3.57^{Δ} | 147.3 ± 2.76^{▴▪} | 150.5 ± 3.38^{▴□} |
| Treat 1 week, 2 hrs | 97.8 ± 4.74^{▴} | 149.7 ± 2.36^{▴} | 140.5 ± 2.67^{▴▪} | 143.3 ± 0.82^{▴□} |
| Treat 1 week, 24 hrs | 99.2 ± 6.83^{▴} | 155.3 ± 2.36^{▴} | 146.5 ± 2.87^{▴▪} | 150.3 ± 0.92^{▴□} |
| Treat 2 weeks, 2 hrs | 96.4 ± 4.08 | 147.4 ± 3.46^{▴} | 138.7 ± 3.01^{▴▪} | 141.6 ± 2.60^{▴} |
| Treat 2 weeks, 24 hrs | 95.7 ± 5.34^{▴} | 156.2 ± 3.25^{▴} | 145.3 ± 2.21^{▴▪} | 148.3 ± 2.75^{▴□} |
| Treat 3 weeks, 2 hrs | 96.4 ± 3.76^{▴} | 149.5 ± 3.24^{▴} | 136.1 ± 4.26^{▴▪} | 139.6 ± 2.32^{▴□} |
| Treat 3 weeks, 24 hrs | 97.6 ± 4.35^{▴} | 154.1 ± 2.08^{▴} | 143.1 ± 3.21^{▴▪} | 145.3 ± 2.20^{▴□} |

| | | | Dendrobium Officinale Powder | |
|---|---|---|---|---|
| | Small Dosage Group | Dual-extraction Group | Group | Model Group |
| Before Treatment | 164.6 ± 4.76 | 165.8 ± 2.75 | 165.0 ± 3.32 | 165.4 ± 3.46 |
| Treat 1 time, 2 hrs | 153.2 ± 3.36^{▴} | 149.6 ± 2.37^{▴} | 149.3 ± 3.18^{▴} | 164.6 ± 4.34 |
| Treat 1 time, 24 hrs | 157.4 ± 3.58^{Δ} | 153.2 ± 2.23^{▴} | 153.8 ± 2.48^{▴} | 164.2 ± 5.37 |
| Treat 1 week, 2 hrs | 156.3 ± 3.46^{▴} | 148.9 ± 1.06^{▴⋆} | 149.9 ± 1.66^{▴⋆} | 165.8 ± 5.18 |
| Treat 1 week, 24 hrs | 158.7 ± 3.87^{Δ} | 151.9 ± 0.96^{▴} | 152.6 ± 1.60^{▴} | 166.7 ± 4.35 |
| Treat 2 weeks, 2 hrs | 154.3 ± 4.05^{▴} | 147.9 ± 2.74^{▴★} | 150.2 ± 2.64^{▴★} | 165.8 ± 4.01 |
| Treat 2 weeks, 24 hrs | 157.2 ± 3.56^{▴} | 152.9 ± 2.71^{▴} | 152.3 ± 1.92^{▴} | 166.5 ± 3.02 |
| Treat 3 weeks, 2 hrs | 152.3 ± 3.76^{▴} | 145.9 ± 2.53^{▴⋆} | 151.8 ± 2.81^{▴★} | 167.4 ± 4.21 |
| Treat 3 weeks, 24 hrs | 156.6 ± 4.05^{▴} | 154.9 ± 3.86^{▴★} | 152.9 ± 3.61^{▴⋆} | 166.4 ± 3.99 |

note:
for the groups compared with the model group, ^{Δ}P < 0.05, ^{▴}P < 0.01; for the large dosage group and medium dosage group compared with the Irbesartan control group, ^{□}P < 0.05, ^{▪}P < 0.01; and for the alcohol-water dual-extraction group and the Dendrobium Officinale powder group compared with the medium dosage group, ^{⋆}P < 0.05, ^{★}P < 0.01.

### Experiment 3

### Experimental Study of Blood Pressure Effect on Normal Rat by the Sample of the Embodiment 1

### Material

Rat: 12 Weeks Old Normal Wistar Rat.

Chinese medicine of the experiment: the sample of the Embodiment 1, dissolved with hot water before use.

Western hypotensive medicine for control: amlodipine besylate tablet (Norvasc), 5 mg/tablet, Pfizer.

Rat blood pressure measurement: BP-98 A type non-invasive tail arterial blood pressure measuring instrument, Softron Co., Ltd. (Japan).

### Method

Grouping treatment: All rats were adaptively fed for a week, measured with blood pressure and weight, and set into groups. The rats are grouped into an Embodiment 1 sample (alcohol-water dual-extraction of Radix Paeoniae Alba with Dendrobium Officinale powder added)-large dosage group of 3.5 g raw medicine/kg body weight and a Western hypotensive medicine amlodipine besylate tablet (Norvasc) group of 5 mg medicine/kg body weight; utilizing gastric infusion; and normal control group receives equal amount of physiological saline. Ten rats for each group, treating for 2 weeks.

Blood pressure measurement: The Rats were pre-heated to 38° C. for 5-10 mins to avoid less blood flow of the tail that affects the pressure measurement. Three measurements are continuously carried out as the rat was in an awake and quiet state. The average value of the 3 measurements was considered as the blood pressure of the rat. The blood pressure is measured prior to the treatment and 2 hours after each administration every week.

### Results

Blood pressure data (x̅±S): See Table 3 and 2 of Experiment 1. After 2 weeks of treatment, the blood pressure of the Embodiment 1 sample (alcohol-water dual-extraction of Radix Paeoniae Alba with Dendrobium Officinale powder added)-large dosage is 139.7±5.82/98.4±4.26 mmHg, which is not significantly different to the blood pressure of the normal control group of 139.6±4.82/97.6±3.76 mmHg (P>0.05). Although the Embodiment 1 sample (alcohol-water dual-extraction of Radix Paeoniae Alba with Dendrobium Officinale powder added) is able to significantly reduce blood pressure for the hypertension cases (SHR) according to the Experiment 1, but the result of the experiment shows that it will not reduce the normal blood pressure.

The blood pressure of the Western hypotensive medicine amlodipine besylate tablet (Norvasc) group is 99.2±5.75/74.5±2.76 mmHg, which is significantly lower than the blood pressure of the normal control group (P<0.01). Orally taking a relatively large dosage of Norvasc can significantly reduce a normal blood pressure.

The above results indicate that utilizing Chinese medicine for hypotensive purpose can avoid hypotension, which is safer.

The dosages used in this experiment for rats corresponding to the equivalent dosages for adults:

The equivalent dosage for adults corresponding to the Embodiment 1 sample (alcohol-water dual-extraction of Radix Paeoniae Alba with Dendrobium Officinale powder added)-large dosage group is about 20 g of raw Dendrobium Officinale/day and 15 g of raw Radix Paeoniae Alba/day. This equals to 5 times of the regular and usual dosage for adults of 7 g (4 g Dendrobium Officinale plus 3 g Radix Paeoniae Alba) daily.

The dosage of the Western hypotensive medicine amlodipine besylate tablet (Norvasc) group equals to 50 mg daily for adults. This equals to 5 times of the regular and usual dosage for adults of 10 mg daily.

### Conclusion

The Embodiment 1 sample (alcohol-water dual-extraction of Radix Paeoniae Alba with Dendrobium Officinale powder added)-large dosage (3.5 g raw medicine/kg body weight) did not trigger hypotensive effect to the normal blood pressure rats, but the Western hypotensive medicine amlodipine besylate tablet (Norvasc) of 5 mg medicine/kg body weight triggered relatively great hypotensive effect to the normal blood pressure rats.

The test results indicate that utilizing Chinese medicine for hypotensive purpose can avoid hypotension, which is safer.

Experiment 3: Table 1. The Effect of the Sample of the Embodiment 1 on the Systolic Pressure of Normal Rats (x̅±S, n=10, Unit: mmHg)

| | Normal | Norvasc | Embodiment 1 |
|---|---|---|---|
| | Control | Large | Sample-Large |
| | Group | Dosage Group | Dosage Group |
| Before | 137.4 ± 6.95 | 137.6 ± 5.46 | 138.7 ± 6.58 |
| Treatment | | | |
| Treated for | 138.2 ± 5.86 | 95.7 ± 4.69^{▴} | 137.8 ± 5.72^{▪} |
| 1 Week | | | |
| Treated for | 139.6 ± 4.82 | 99.2 ± 5.75^{▴} | 139.7 ± 5.82^{▪} |
| 2 Weeks | | | |

note:
for the groups compared with the normal group,
ΔP < 0.05,
^{▴}P < 0.01;
for the Embodiment 1 sample group compared with the Norvasc group,
□P < 0.05,
^{▪}P < 0.01.

Experiment 3: Table 2. The Effect of the Sample of the Embodiment 1 on the Diastolic Pressure of Normal Rats (x̅±S, n=10, Unit: mmHg)

| | Normal | Norvasc | Embodiment 1 |
|---|---|---|---|
| | Control | Large Dosage | Sample-Large |
| | Group | Group | Dosage Group |
| Before | 98.4 ± 5.62 | 99.2 ± 4.87 | 97.6 ± 5.84 |
| Treatment | | | |
| Treated for | 99.3 ± 4.52 | 71.8 ± 3.76^{▴} | 98.5 ± 3.54^{▪} |
| 1 Week | | | |
| Treated for | 97.6 ± 3.76 | 74.5 ± 2.76^{▴} | 98.4 ± 4.26^{▪} |
| 2 Weeks | | | |

note:
for the groups compared with the normal group,
ΔP < 0.05,
^{▴}P < 0.01;
for the Embodiment 1 sample group compared with the Norvasc group,
□P < 0.05,
^{▪}P < 0.01.

### Experiment 4

### Experimental Study of Hypotensive Effect on Hereditary Hypertension Rat (SHR) by the Samples of the Embodiment 1, Embodiment 2, and Embodiment 3

### Material

Rats: old age (over 48 weeks old) hereditary hypertension rats (SHR) and the normal controls were Wistar rats.

Chinese medicine 1 of the experiment: the sample of the Embodiment 1 (6 servings of alcohol-water dual-extraction of Radix Paeoniae Alba with 8 servings of Dendrobium Officinale powder added), dissolved with hot water before use.

Chinese medicine 2 of the experiment: the sample of the Embodiment 2 (2 servings of alcohol-water dual-extraction of Radix Paeoniae Alba with 8 servings of Dendrobium Officinale powder added), dissolved with hot water before use.

Chinese medicine 3 of the experiment: the sample of the Embodiment 3 (8 servings of alcohol-water dual-extraction of Radix Paeoniae Alba with 2 servings of Dendrobium Officinale powder added), dissolved with hot water before use.

Rat blood pressure measurement: BP-98 A type non-invasive tail arterial blood pressure measuring instrument, Softron Co., Ltd. (Japan).

### Method

Grouping treatment: All rats were adaptively fed for a week, measured with blood pressure and weight, and set into groups. The SHR rats are grouped into:

Embodiment 1 sample (6 servings of alcohol-water dual-extraction of Radix Paeoniae Alba with 8 servings of Dendrobium Officinale powder added)-medium dosage group: 1.4 g of raw medicine/kg body weight medium (0.8 g of raw Dendrobium Officinale/kg body weight+0.6 g of raw Radix Paeoniae Alba/kg body weight);

Embodiment 2 sample (2 servings of alcohol-water dual-extraction of Radix Paeoniae Alba with 8 servings of Dendrobium Officinale powder added)-medium dosage group: 1.4 g of raw medicine/kg body weight medium (1.12 g of raw Dendrobium Officinale/kg body weight+0.28 g of raw Radix Paeoniae Alba/kg body weight); and

Embodiment 3 sample (8 servings of alcohol-water dual-extraction of Radix Paeoniae Alba with 2 servings of Dendrobium Officinale powder added)-medium dosage group: 1.4 g of raw medicine/kg body weight medium (0.28 g of raw Dendrobium Officinale/kg body weight+1.12 g of raw Radix Paeoniae Alba/kg body weight).

These three treatment groups apply gastric infusion and the hypertension model control group receives equal amount of physiological saline through gastric infusion as well. Ten rats for each group, treating for 3 weeks.

Blood pressure measurement: The Rats were pre-heated to 38° C. for 5-10 mins to avoid less blood flow of the tail that affects the pressure measurement. Three measurements are continuously carried out as the rat was in an awake and quiet state. The average value of the 3 measurements was considered as the blood pressure of the rat. The blood pressure is measured prior to the treatment and 2 hours and 24 hours after each administration every week.

### Results

1. Two hours after administration, blood pressure data (x̅±S): See Table 1 and 2 of Experiment 4.

When treated for 3 weeks, the blood pressure of the sample of the Embodiment 1 (6 servings of the alcohol-water dual-extraction of Radix Paeoniae Alba with 8 servings of Dendrobium Officinale powder added) group was 170.3±2.64/140.1±3.21 mmHg, the blood pressure of the sample of the Embodiment 2 (2 servings of the alcohol-water dual-extraction of Radix Paeoniae Alba with 8 servings of Dendrobium Officinale powder added) group was 181.2±2.13/149.8±2.13 mmHg and the blood pressure of the sample of the Embodiment 3 (8 servings of the alcohol-water dual-extraction of Radix Paeoniae Alba with 2 servings of Dendrobium Officinale powder added) group was 172.4±3.67/141.2±3.58 mmHg, which were all significantly lower than the blood pressure of the hypertension model control group of 217.5±4.61/166.6±5.21 mmHg (P<0.01). The proportions of the Chinese medicines are allocated differently in these three treating groups, while the total raw medicine dosages are the same. Based on the data of the blood pressure of 2 hours after administration, the blood pressure of the Embodiment 2 sample group (2 servings of the alcohol-water dual-extraction of Radix Paeoniae Alba with 8 servings of the Dendrobium Officinale powder added) is significantly higher than it of the Embodiment 1 sample group (6 servings of the alcohol-water dual-extraction of Radix Paeoniae Alba with 8 servings of the Dendrobium Officinale powder added) (P<0.01). This preliminarily indicates that when the alcohol-water dual-extraction of the Radix Paeoniae Alba is in a proper proportion (e.g. 4/1); it has a fast hypotensive effect (fast hypotensive, but short duration).

2. Two Hours after Administration, Blood Pressure Data (x̅±S): See Table 1 and 24 of Experiment 4.

When treated for 3 weeks, the blood pressure of the Embodiment 1 sample group was 190.2±1.55/147.9±2.55 mmHg, the blood pressure of the Embodiment 2 sample group was 192.7±2.64/150.1±3.21 mmHg, and the blood pressure of the Embodiment 3 sample group was 200.8±3.72/154.1±2.13 mmHg, which were all significantly lower than the blood pressure of the hypertension model control group of 216.8±3.97/165.8±3.10 mmHg (P<0.01). The proportions of the Chinese medicines are allocated differently in these three treating groups, while the total raw medicine dosages are the same. Based on the data of the blood pressure of 24 hours after administration, the blood pressure of the Embodiment 3 sample group (8 servings of the alcohol-water dual-extraction of Radix Paeoniae Alba with 2 servings of the Dendrobium Officinale powder added) is significantly higher than it of the Embodiment 1 sample group (6 servings of the alcohol-water dual-extraction of Radix Paeoniae Alba with 8 servings of the Dendrobium Officinale powder added) (P<0.05). This preliminarily indicates that when the Dendrobium Officinale is in a proper proportion (e.g. 4/1), it has an effect of stabilizing the decreased blood pressure for 24 hours (hypotensive peak will not be too low and the fluctuation of the blood pressure is low)

3. The Ratio of the Blood Pressure Reductions of the 24 Hours after Administration and 2 Hours after Administration: See Table 1 and 2 of Experiment 4.

When treated for 3 weeks, the ratios of the blood pressure reduction differences between before and after treatment as well as the hypotensive valley differences were:

The reduction of the Embodiment 1 sample group is systolic pressure reduction-24 hours after administration/2 hours after administration: −21.30/−41.90 mmHg, ratio of the hypotensive valley and peak: 0.5083; and diastolic pressure reduction-24 hours after administration/2 hours after administration: −14.33/−21.67 mmHg, ratio of the hypotensive valley and peak: 0.6613. Embodiment 1 sample group showed relatively larger reduction among the three groups, with the hypotensive valley peak ratio of 24 hours and 2 hours after administration larger than 50%, which hypotensive effect was more stable.

The reduction of the Embodiment 2 sample group is systolic pressure reduction-24 hours after administration/2 hours after administration: −20.13/−32.90 mmHg, ratio of the hypotensive valley and peak: 0.6119; and diastolic pressure reduction-24 hours after administration/2 hours after administration: −13.02/−14.52 mmHg, ratio of the hypotensive valley and peak: 0.8930. Embodiment 2 sample group showed relatively smallest blood pressure fluctuation during the reduction among the three groups, with the hypotensive valley peak ratio of 24 hours and 2 hours after administration larger than 60%, which hypotensive effect was the most stable among the three groups. However, its blood pressure reduction is relatively small.

The reduction of the Embodiment 3 sample group is systolic pressure reduction-24 hours after administration/2 hours after administration: −13.75/−43.52 mmHg, ratio of the hypotensive valley and peak: 0.3159; and diastolic pressure reduction-24 hours after administration/2 hours after administration: −10.13/−23.12 mmHg, ratio of the hypotensive valley and peak: 0.4381. Embodiment 3 sample group showed largest hypotensive peak value among the three groups, with the hypotensive peak value valley peak ratio of 24 hours and 2 hours after administration smaller than 50%, which provided fast hypotensive effect.

Based on the above findings, the hypotensive extent and blood pressure stability of the Embodiment 1 sample are both better, which may be utilized as a major and common hypotensive medicine; the hypotensive speed and extent of the Embodiment 2 sample are both mild, while the blood pressure stability thereof is relatively good, which may be utilized for stable hypotensive or blood pressure maintenance purposes; and both the hypotensive extent and speed of the Embodiment 3 sample are great, while the effect thereof is short, which may be suitable for a short term hypotensive purpose within a day. These samples of the embodiments with various formulas all have significant hypotensive effect as well as diverse characteristics.

The dosages used in this experiment for rats corresponding to the equivalent dosages for adults:

The raw medicine dosage of the Embodiment 1 sample (6 servings of alcohol-water dual-extraction of Radix Paeoniae Alba with 8 servings of Dendrobium Officinale powder added) is 1.4 g/kg body weight (0.8 g of raw Dendrobium Officinale/kg body weight+0.6 g of raw Radix Paeoniae Alba/kg body weight). The corresponding total raw medicine dosage for adults is about 14 g (8 g of Dendrobium Officinale and 6 g of Radix Paeoniae Alba) daily.

The raw medicine dosage of the Embodiment 2 sample (2 servings of alcohol-water dual-extraction of Radix Paeoniae Alba with 8 servings of Dendrobium Officinale powder added) is 1.4 g/kg body weight (1.12 g of raw Dendrobium Officinale/kg body weight+0.28 g of raw Radix Paeoniae Alba/kg body weight). The corresponding total raw medicine dosage for adults is about 14 g (11.2 g of Dendrobium Officinale and 2.8 g of Radix Paeoniae Alba) daily.

The raw medicine dosage of the Embodiment 3 sample (8 servings of alcohol-water dual-extraction of Radix Paeoniae Alba with 2 servings of Dendrobium Officinale powder added) is 1.4 g/kg body weight (0.28 g of raw Dendrobium Officinale/kg body weight+1.12 g of raw Radix Paeoniae Alba/kg body weight). The corresponding total raw medicine dosage for adults is about 14 g (2.8 g of Dendrobium Officinale and 11.2 g of Radix Paeoniae Alba) daily.

### Conclusion

All the sample of the Embodiment 1 (6 servings of the alcohol-water dual-extraction of Radix Paeoniae Alba with 8 servings of Dendrobium Officinale powder added), the sample of the Embodiment 2 (2 servings of the alcohol-water dual-extraction of Radix Paeoniae Alba with 8 servings of Dendrobium Officinale powder added), and the sample of the Embodiment 3 (8 servings of the alcohol-water dual-extraction of Radix Paeoniae Alba with 2 servings of Dendrobium Officinale powder added) with gastric infusion of the raw dosage of 1.4 g/kg body weight showed significant hypotensive effect.

This preliminarily indicates that only when the alcohol-water dual-extraction of the Radix Paeoniae Alba is in a proper proportion (e.g. 4/1); it is able to facilitate a fast hypotensive effect (fast hypotensive, but short duration). Only when the Dendrobium Officinale is in a proper proportion (e.g. 4/1), it has an effect of stabilizing the decreased blood pressure for 24 hours (hypotensive peak will not be too low and the fluctuation of the blood pressure is low.)

The hypotensive extent and blood pressure stability of the Embodiment 1 sample are both better, which may be utilized as a major and common hypotensive medicine; the hypotensive speed and extent of the Embodiment 2 sample are both mild, while the blood pressure stability thereof is relatively good, which may be utilized for stable hypotensive or blood pressure maintenance purposes; and both the hypotensive extent and speed of the Embodiment 3 sample are great, while the effect thereof is short, which may be suitable for a short term hypotensive purpose within a day. These samples of the embodiments with various formulas all have significant hypotensive effect as well as diverse characteristics.

Experiment 4: Table 1. The Effect of the Samples of the Embodiment 1, Embodiment 2, and Embodiment 3 on the Systolic Pressure of SHR Rats (x̅±S, n=10, Unit: Mmhg)

| | Embodiment 1 | Embodiment 2 | Embodiment 3 | |
|---|---|---|---|---|
| | Sample Group | Sample Group | Sample Group | Model Group |
| Before Treatment | 214.5 ± 3.66 | 215.7 ± 5.72 | 215.7 ± 4.48 | 214.8 ± 5.76 |
| Treat 1 time, 2 hrs | 175.9 ± 3.04^{▴} | 183.9 ± 2.56^{▴⋆} | 172.2 ± 3.21^{▴} | 215.8 ± 4.32 |
| Treat 1 time, 24 hrs | 195.9 ± 2.65^{▴} | 198.7 ± 3.87^{▴} | 203.7 ± 2.12^{▴⋆} | 216.2 ± 4.44 |
| Treat 1 week, 2 hrs | 173.7 ± 2.76^{▴} | 183.8 ± 2.01^{▴★} | 172.8 ± 5.21^{▴} | 216.4 ± 4.35 |
| Treat 1 week, 24 hrs | 194.3 ± 2.47^{▴} | 196.2 ± 3.89^{▴} | 203.2 ± 2.57^{▴⋆} | 216.2 ± 3.65 |
| Treat 2 weeks, 2 hrs | 170.5 ± 1.98^{▴} | 182.3 ± 2.35^{▴★} | 171.3 ± 4.37^{▴} | 217.2 ± 4.57 |
| Treat 2 weeks, 24 hrs | 192.4 ± 2.57^{▴} | 194.7 ± 3.71^{▴} | 200.1 ± 3.12^{▴⋆} | 216.5 ± 3.42 |
| Treat 3 weeks, 2 hrs | 170.3 ± 2.64^{▴} | 181.2 ± 2.13^{▴★} | 172.4 ± 3.67^{▴} | 217.5 ± 4.61 |
| Treat 3 weeks, 24 hrs | 190.2 ± 1.55^{▴} | 192.7 ± 2.64^{▴} | 200.8 ± 3.72^{▴⋆} | 216.8 ± 3.97 |
| Average Blood | 172.60 | 182.80 | 172.18 | |
| Pressure of 2 hrs | | | | |
| Average Blood | 193.20 | 195.58 | 201.95 | |
| Pressure of 24 hrs | | | | |
| Average Reduction of | −41.90 | −32.90 | −43.52 | |
| 2 hrs | | | | |
| Average Reduction of | −21.30 | −20.13 | −13.75 | |
| 24 hrs | | | | |
| Ratio of the | 0.5083 | 0.6119 | 0.3159 | |
| hypotensive peak and | | | | |
| valley | | | | |

note:
for the groups compared with the model group, ΔP < 0.05, ^{▴}P < 0.01; for the Embodiment 2 group, Embodiment 3 group compared with the Embodiment 1 group, ^{⋆}P < 0.05, ^{★}P < 0.01.

Experiment 4: Table 2. The Effect of the Samples of the Embodiment 1, Embodiment 2, and Embodiment 3 on the Diastolic Pressure of SHR Rats (x̅±S, n=10, Unit: Mmhg)

| | Embodiment 1 | Embodiment 2 | Embodiment 3 | |
|---|---|---|---|---|
| | Sample Group | Sample Group | Sample Group | Model Group |
| Before Treatment | 163.7 ± 3.45 | 164.6 ± 3.37 | 165.1 ± 4.79 | 165.7 ± 4.82 |
| Treat 1 time, 2 hrs | 145.6 ± 2.71^{▴} | 153.1 ± 2.81^{▴★} | 142.4 ± 3.28^{▴} | 164.3 ± 3.57 |
| Treat 1 time, 24 hrs | 150.8 ± 2.21^{▴} | 153.3 ± 4.25^{▴} | 155.4 ± 3.58^{▴} | 165.3 ± 3.15 |
| Treat 1 week, 2 hrs | 142.1 ± 1.80^{▴} | 149.2 ± 1.67^{▴★} | 142.2 ± 4.51^{▴} | 166.7 ± 4.27 |
| Treat 1 week, 24 hrs | 149.6 ± 1.34^{▴} | 151.6 ± 1.91^{▴} | 155.1 ± 2.10^{▴⋆} | 166.2 ± 3.48 |
| Treat 2 weeks, 2 hrs | 140.3 ± 2.75^{▴} | 148.2 ± 2.10^{▴★} | 142.1 ± 4.11^{▴} | 165.4 ± 3.47 |
| Treat 2 weeks, 24 hrs | 149.2 ± 2.65^{▴} | 151.3 ± 3.76^{▴} | 155.3 ± 2.54^{▴⋆} | 165.1 ± 3.81 |
| Treat 3 weeks, 2 hrs | 140.1 ± 3.21^{▴} | 149.8 ± 2.13^{▴★} | 141.2 ± 3.58^{▴} | 166.6 ± 5.21 |
| Treat 3 weeks, 24 hrs | 147.9 ± 2.55^{▴} | 150.1 ± 3.21^{▴} | 154.1 ± 2.13^{▴⋆} | 165.8 ± 3.10 |
| Average Blood | 142.03 | 150.08 | 141.98 | |
| Pressure of 2 hrs | | | | |
| Average Blood | 149.38 | 151.58 | 154.98 | |
| Pressure of 24 hrs | | | | |
| Average Reduction | −21.67 | −14.52 | −23.12 | |
| of 2 hrs | | | | |
| Average Reduction | −14.33 | −13.02 | −10.13 | |
| of 24 hrs | | | | |
| Ratio of the | 0.6613 | 0.8930 | 0.4381 | |
| hypotensive peak | | | | |
| and valley | | | | |

note:
for the groups compared with the model group, ΔP < 0.05, ^{▴}P < 0.01; for the Embodiment 2 group, Embodiment 3 group compared with the Embodiment 1 group, ^{⋆}P < 0.05, ^{★}P < 0.01.

### Experiment 5

### Clinical Observation of the Embodiment 1 Sample (Radix Paeoniae Alba and Dendrobium Officinale) Treating Hypertension

### Research Medicine

The Embodiment 1 sample (6 servings of alcohol-water dual-extraction of Radix Paeoniae Alba with 8 servings of Dendrobium Officinale powder added): Radix Paeoniae Alba and Dendrobium Officinale tablet. Each tablet contains: 0.444 g of Dendrobium Officinale and 0.333 g of Radix Paeoniae Alba. It was orally taken, 3 tablets each time, and 3 times daily. Daily dosage contains 4 g of Dendrobium Officinale and 3 g of Radix Paeoniae Alba.

### Clinical Information

In recent special studies, mild and moderate hypertension patients in the outpatient service were observed, in which patients who were willing to accept long-term visit were collected for the blood pressure tracking and observations. Thirty-one complete cases with the blood pressure of each reported for more than 3 days per week, more than two times of measurement per day, and a continual observation for at least 4 weeks for before and after treatment, are reported as follows:

These 31 patients include 13 males and 18 females with age of 57.7±7.2. Twenty-one of them had mild hypertension, while 9 of them had moderate hypertension. Twenty-four of them had not used Western hypotensive medicine for one month before entered this observation research.

### Observation Method

Blood pressure measurement and assessment: measuring blood pressure in the morning, noon, and evening daily, wherein the household electronic sphygmomanometer had been compared with the mercury column sphygmomanometer for the results when bought and used only if the results were similar. The blood pressures were measured many times per day during the entire process. The average of the blood pressures measured in a day was considered the blood pressure of the day. The average of the blood pressures of the days of a week was considered the blood pressure of the week for comparing the differences before and after treatment as well as the blood pressure stability during the treatment. This avoids deviations of the blood pressure assessments rendered by the blood pressure fluctuations of a day or occasional fluctuation in the measurement day. After multiple days of measurements of multiple times per day, the actual daily blood pressure of the patients were confirmed, so as for avoiding the “white coat hypertension” due to the presence of the patients in the hospital. This also avoided the drawback that a one-time blood pressure check in the hospital might not well indicate and cover the patient's daily blood pressure.

Three to five days of blood pressure measurements (multiple times per day) were conducted before treatment, so as for calculating and obtaining the average blood pressures before treatment as a pre-treatment blood pressure baseline. After the beginning of the treatment, the average level of blood pressure per week was calculated, and the hypotensive curative effect was evaluated, so as to avoid the contingency error of single blood pressure or single-day blood pressure from affecting the curative effect evaluation.

Administration management: If 2 weeks of blood pressure after administration was less than 135/85 mmHg, an Irbesartan 75 mg tablet (equivalent to a half-piece of 150 mg tablet) can be added, and the amount of the Western medicine added in the observation period would be calculated at the end of the observation.

In the course of medicine taking, the blood pressures of some patients were first reduced, but then raised again due to work fatigue or some other reasons. In such cases, the dosage of the Chinese medicine Radix Paeoniae Alba and Dendrobium Officinale tablet can be added into 6 tablets/time and 3 times/day. Based on the blood pressure reduction conditions of the patient, the dosage would be restored into a common maintenance amount (small dosage). That is, a step dosage can be utilized according to the blood pressure fluctuation.

If the blood pressure in the observation period falls below the criteria, the dosage of the Chinese medicine could be reduced, and the dosage of the decrease in the observation period would be calculated at the end of the observation period.

### Observation Results

The condition that Western medicine Irbesartan was added: Three of the patients used only one Chinese medicine showed slow blood pressure reduction that their blood pressures failed to reach the criteria after 2 weeks; therefore 75 mg/day of Irbesartan (small dosage) was applied, and the blood pressures all reached the standard thereafter. This is 10% of the cases.

Chinese medicine reduction: The blood pressures of 3 of the patients had reached the criteria during the treatment that their dosages were gradually reduced. These three patients averagely took 4 tablets per day by the third week. It cut the planned dosage into half, but was still able to maintain the target blood pressure.

Chinese medicine increase: The blood pressures of 5 of the patients had fluctuated and elevated during the treatment due to business trip and work fatigue that their dosage were temporarily increased into 6 of the Radix Paeoniae Alba and Dendrobium Officinale tablets each time and 3 times per day (3 tablets each time is considered small dosage, while 6 tablets each time is considered medium dosage). These doubled dosages (medium dosage) were utilized for only 1-2 days each.

Blood pressure states: see Table 1. The blood pressures were significantly decreased after treatment (P<0.01/P<0.05).

Experiment 5: Table 1. The Radix Paeoniae Alba and Dendrobium Officinale tablet in treating 31 cases of hypertension-Blood pressures before and after treatment (unit: mmHg)

| | Before | Treated | |
|---|---|---|---|
| | Treatment | for 4 Week | Reduction |
| Systolic Pressure | 145.55 ± 9.27 | 122.30 ± 6.45^{▴} | 23.25 |
| Diastolic Pressure | 90.76 ± 5.32 | 80.56 ± 5.51^{Δ} | 10.20 |

note.
t-test for the before and after treatment,
^{▴}P < 0.01,
^{Δ}P < 0.05.

Side effect: no obvious side effect or toxicity has been observed. (Both Radix Paeoniae Alba and Dendrobium Officinale are on the official list of the health-care food materials of China, which shall be safe and non-toxic.)

Observation of the discontinuation: after the observation of some of the cases had finished, their blood pressures is completely normal and they discontinued the Chinese medicine, but was continually visited. Five patients were observed normal blood pressure after the withdrawal of the medicine for 12 months and without utilizing Western hypotensive medicine.

### Discussion and Conclusion

According to the clinical test data on the instructions of Irbesartan (Aprovel®) (approved Mar. 10, 2007 and modified on Feb. 12, 2015): adult daily dosage of 150-300 mg, sitting and lying blood pressure, the average hypotensive valley value is 8-13/5-8 mmHg and the ratio of the hypotensive valley and peak is 60-80%. If the intermediate values of all these data were taken, the hypotensive extent of the Irbesartan tablet in the hypotensive peak can be calculated and obtained: systolic pressure of 10.5/diastolic pressure of 6.5 mmHg/0.7=systolic pressure of 15.00/diastolic pressure of 9.29 mmHg. The average hypotensive extent between the peak and valley of the Chinese medicine Radix Paeoniae Alba and Dendrobium Officinale tablet (small dosage: the average raw medicine of 4 g of the Dendrobium Officinale and 3 g of the Radix Paeoniae Alba) of 23.25/10.20 mmHg (blood pressures measured in the morning, noon, and evening daily) has surpassed the hypotensive extent between the peak and valley of the regular dosage of Irbesartan tablet (150-300 mg daily for adults) of 15.00/9.29 mmHg. (Three of the thirty-one of the patients of the present clinical observation had additionally utilized a minor dosage (75 mg/day) of Irbesartan, but their hypotensive extents surpassed it of the full dosage according to the instructions of Irbesartan.)

The Chinese medicine Radix Paeoniae Alba and Dendrobium Officinale may effectively reduce the blood pressure of mild and moderate hypertension patients. When it was utilized for blood pressure control only, more than 80% of the patients had reached the criteria of normal blood pressure.

The proportion of the patients that required additional Western medicine Irbesartan was little and only very small dosage added was sufficient for them to achieve the criteria of normal blood pressure.

The prepared Chinese medicine of Radix Paeoniae Alba and Dendrobium Officinale may be solely utilized in treating hypertension or be utilized along with hypotensive medicines like Western medicine Irbesartan for enhancing the hypotensive curative effect, enhancing the sensitivity of the Western hypotensive medicine, and reducing the dosage of the Western hypotensive medicine. This may also provide a solution to increase the hypotensive curative effect of combined pharmacotherapy for treating difficult hypertension.

The mechanism of the prepared traditional Chinese medicine Radix Paeoniae Alba and Dendrobium Officinale for treating hypertension may have been related to body constitution adjustment thereby. Therefore, the mechanism of the blood pressure reduction thereof may be different from it of Western hypotensive medicine that blocks the segment of blood pressure increase. As a result, the curative effect of the Chinese medicine lasts longer and is more meaningful for health. It may have a long-term blood pressure stabilizing effect.

It is worth mentioning that:

In view of the homology of the traditional Chinese medicine and the health-care product, most Chinese medicine has no significant effect on normal blood pressure and therefore is safer in being used as a health-care food. In most cases, a Chinese medicine and a health-care food only differ in the national management categories, while the substantive contents of them are basically or even completely identical. The product provided by the present invention also belongs to this case that it can both be used as a Chinese medicine for treating hypertension and be used as a daily health-care food for persons who have mild hypertension or high normal blood pressure, while the methods of both uses are the same.

What is claimed is:

1. An application method of Radix Paeoniae Alba and Dendrobium Officinale for preparing drug for treatment of hypertension, comprising the following steps:
(1) 8 servings of Dendrobium Officinale and 2-32 servings of Radix Paeoniae Alba according to the requirements of Chinese Pharmacopoeia (2015), wherein serving refers to a serving by weight;
(2) grinding the Radix Paeoniae Alba into powder and carrying out alcohol extraction: adding 7 times of the raw medicine amount of 70% ethanol for reflux extraction for 1 hour, or leaching at 60° C. for 24 hours, wherein the Radix Paeoniae Alba is totally extracted twice, and the ethanol is collected;
(3) water extracting the alcohol extracted residual:
a. extracting for two times with hot water at 100° C., and combining the two extracting solutions; or
b. extracting by using hot water at a gradient temperature twice and combining the two extracting solutions, wherein the water temperature is 100° C. for the first water extraction, and the water temperature is 121° C. for the second water extraction, wherein the water extraction time of the two times is 1 hour each;
(4) respectively concentrating the alcohol and water extracting solutions and combining the concentrated extracting solutions; and
(5) grinding the Dendrobium Officinale into powder, and adding into the combined extracting solution; and adding auxiliary materials according to a pharmaceutically conventional processing method so as to prepare a traditional Chinese medicine tablet, suspension particles, pill, powder, oral liquid or capsule to provide the medicine for treating hypertension.

2. The application method, as recited in claim 1, comprising the step of: taking the prepared traditional Chinese medicine tablet, particles or suspension particles, pill, powder, oral liquid or capsule orally, wherein the daily dosage of an adult is equivalent to 2.5 g to 40 g of the raw medicine material.

3. The application method, as recited in claim 1 as a supplement and health-care food, comprising the step of: taking the prepared traditional Chinese medicine tablet, particles or suspension particles, pill, powder, oral liquid or capsule as a supplement and health-care food orally, wherein the daily dosage of an adult is equivalent to 2.5 g to 40 g of the raw medicine material.

4. The application method, as recited in claim 1, wherein the weight servings of the Dendrobium Officinale and the Radix Paeoniae Alba are 4:3, wherein when the medicine is orally taken, the daily dosage of an adult is equivalent to 7 g to 14 g of the raw medicine.

5. The application method, as recited in claim 1, wherein the weight servings of the Dendrobium Officinale and the Radix Paeoniae Alba are 4:1, wherein when the medicine is orally taken, the daily dosage of an adult is equivalent to 5 g to 15 g of the raw medicine.

6. The application method, as recited in claim 1, wherein the weight servings of the Dendrobium Officinale and the Radix Paeoniae Alba are 1:4, wherein when the medicine is orally taken, the daily dosage of an adult is equivalent to 5 g to 15 g of the raw medicine.
